(19) United States
(12) Patent Application Publication
(10) Pub. No.: US 2014/0038847 A1
(43) Pub. Date: Feb. 6, 2014

(54) SCREENING AND CULTURING DEVICE AND METHODS FOR THE USE THEREOF

(71) Applicant: Microstem, Inc., San Diego, CA (US)

(72) Inventors: Marie Zhang, San Diego, CA (US); Justin Bingham, Coronado, CA (US); Naira Serobyan, San Diego, CA (US)

(73) Assignee: Microstem, Inc., San Diego, CA (US)

(21) Appl. No.: 13/891,826

(22) Filed: May 10, 2013

Related U.S. Application Data
(63) Continuation of application No. PCT/US2011/060269, filed on Nov. 10, 2011.
(60) Provisional application No. 61/412,771, filed on Nov. 11, 2010.

(51) Int. Cl. G01N 33/50

(52) U.S. Cl. CPC G01N 33/5011; G01N 33/5026
USPC 506/10; 506/18; 506/30

(57) ABSTRACT

In accordance with one aspect of the present invention, screening and culturing devices have been developed which are useful for the identification of media which support cell viability, growth and/or proliferation, and transformation and/or differentiation. In a further aspect, screening and culturing methods employing the invention screening and culturing devices have been developed. In a still further aspect, methods for making invention screening and culturing devices have been developed.

### CROSS-REFERENCES TO RELATED APPLICATIONS

This application is a continuation of PCT Application No. PCT/US2011/060269, filed Nov. 10, 2011 which claims the benefit of U.S. Provisional Application No. 61/412,771, filed Nov. 11, 2010, the contents of which are incorporated herein by reference in their entirety for all purposes.

### FIELD OF THE INVENTION

The present invention relates to screening and culturing devices which are useful for the identification of media which support cell viability, growth and/or proliferation. In a further aspect, the invention relates to screening and culturing methods employing the invention screening and culturing devices. In a still further aspect, the invention relates to methods for making invention screening and culturing devices.

### BACKGROUND OF THE INVENTION

Cells in organisms reside in an environment consisting of extra cellular matrices (ECMs) to support cell viability, growth and/or proliferation, and transformation and/or differentiation. ECMs contain any number of components native to that organism, including but not limited to extra cellular matrix proteins (ECMPs), signaling molecules, cytokines, glycans, proteoglycans, and cell adhesion molecules. In order to grow cells in vitro, appropriate ECMs should be identified for every type of cell to promote optimal cell culture in dishes, flasks and other standard cell culture vessels. To date, general practice involves coating or purchasing pre-coated standard cell culture vessels with ECMs, such as Collagen I, III, IV, V, VI, Fibronectin, Laminin, Vitronectin, Elastin, Tenascin, Decorin, etc. individually or in combination to support appropriate cell growth and functioning in vitro. However, finding the appropriate ECMs or combination of ECMs for proper cell growth and behavior in vitro is a trial-and-error process. This multi-step process is time consuming, and requires considerable amounts of ECM components and cells which are costly.

For example, traditional in vitro models used for the development of anti-cancer drugs are based on the monolayer culture of cells, which has limited in vivo efficacy. For example, it is observed that culturing MCF-7 breast cancer cells within the three-dimensional (3D) environment of the microwells alone has an effect on the response of these cells to the anti-cancer drug, paclitaxel, resulting in a reduction of cell death in comparison to cells cultured on flat substrates.

Since paclitaxel, originally isolated from Taxus brevifolia (the pacific yew), is one of the most active chemotherapeutic agents known, with activity against a wide panel of solid tumors (including urothelial, breast, lung, and ovarian cancers), an understanding of the mechanism of the effect of paclitaxel on inducing tumor cell apoptosis and the discovery of new ways to enhance the effect of paclitaxel will be useful for improving the therapeutic efficiency of this drug. Unfortunately, the exact mechanism by which paclitaxel induces apoptosis is not clear.

When MCF7 cells, for example, are treated with paclitaxel, the cells display morphological alterations typical of adherent cells undergoing apoptosis, i.e., they become rounded, condensed and detached from the dish. Paclitaxel affects rapidly dividing cells by stabilizing microtubules and as a result, interferes with the normal breakdown of microtubules during cell division. The end result is defects in spindle assembly, chromosome segregation and cell division. This inability of the chromosomes to achieve metaphase spindle configuration leads to a mitotic block in which there is prolonged activation of the mitotic checkpoint with the subsequent triggering of apoptosis. Paclitaxel binds to the beta-tubulin subunit. There is some indication in recent publications of differential expression of the beta-tubulin gene depending on the makeup of the ECM.

A number of 2D and 3D cell culture platforms have been developed to study the therapeutic effect of chemotherapeutic drugs such as paclitaxel. However these platforms most often are not amenable to screening large numbers of combinations on variety of ECMs and require large amounts of cells.

Another example of the makeup of the ECM influencing cellular behavior is in the area of stem cell differentiation. While most stem cell differentiation studies have been focused on growth factors, recently convincing studies have demonstrated the importance of the extracellular matrix molecules on regulation of stem cell fate. For example, differentiation of mesenchymal stem cells (MSCs) to tissue specific cells is mediated by a complex series of cell-cell and cell-extracellular matrix interactions. Mesenchymal stem cells cultured on collagen type I have been shown to preferentially undergo osteogenic differentiation, while MSCs exposed to collagen types I and II are prompted to undergo chondrogenic differentiation.

Differentiation of human stem cells into cardiomyocytes and stem cell transplantation to repair injured myocardium are new frontiers in cardiovascular research. Bone Marrow (BM) cells possess unique properties that make them a suitable candidate for regeneration of cardiac tissue. Undifferentiated bone marrow mesenchymal stem cells (MSCs) are known to have the ability to differentiate multiple cell lineages including osteoblasts, adipocytes, chondrocytes and cardiomyocytes under appropriate culture conditions in vitro. In order to enhance engraftment efficiency of transplanted mesenchymal stem cells, and subsequently improve clinical efficacy of cell therapy, it would be desirable for multipotent bone marrow MSCs to be differentiated to some degree toward a cardiomyogenic lineage in vitro before cell transplantation.

Yet another example of the makeup of the ECM influencing cellular behavior is the occurrence of a cell transformation event when cancer cells undergo epithelial mesenchymal transition (EMT) during invasion and metastasis. It is well documented that this transition can be initiated in vitro by the addition of TGF-β (transforming growth factor-beta) and is marked by the reduced expression of the epithelial marker, e-cadherin, and increased expression of the mesenchymal marker, vimentin.

Drug discovery and basic research programs often face the challenges of translating information obtained from in vitro experiments to the in vivo setting. A major contributing factor is the difficulty of using an in vitro cell culture environment to mimic events as they naturally occur inside the body. One aspect often underutilized in cellular assay development is an evaluation of the effect of extra cellular matrices (ECMs) on cell growth and function.

### BRIEF DESCRIPTION OF THE INVENTION

In one aspect of the present invention, there are provided combinatorial extracellular matrix (ECM) screening and culturing devices comprising a suitable support coated with a hydrogel, on which a plurality of spots comprising one or more ECM components are printed thereon,

wherein:
the concentration of ECM component(s) per spot falls in the range of about 0.01 mg/ml up to about 1 mg/ml,
each ECM component is printed in replicates of at least 3 up to about 20,
the resulting ECM spots have a minimum diameter to allow attachment of at least one cell thereto (typically in the range of about 50 up to about 1000 μm), and
the center-to-center distance between spots is sufficient to preclude overlap of the spots (typically the distance is at least about 100 μm).

In another aspect of the present invention, methods are provided for making a screening and culturing device as described herein, said methods comprising printing a plurality of ECM components on a suitable support material.

In accordance with yet another aspect, there are also provided methods of screening a plurality of extracellular matrix (ECM) components to identify those which support cell viability, growth and/or proliferation, and transformation and/or differentiation, said method comprising:
applying cells to a screening and culturing device as described herein,
assaying cell morphology and/or behavior upon incubation of said cell, and
identifying those ECM components which support cell viability, growth and/or proliferation, and transformation and/or differentiation.

In accordance with still another aspect, there are also provided methods of culturing cells on a plurality of extracellular matrix (ECM) components which support cell viability, growth and/or proliferation, said method comprising:
applying cells to a screening and culturing device as described herein,
assaying cell morphology and/or behavior upon incubation of said cell, and
identifying those ECM components which support cell viability, growth and/or proliferation.

Screening employing the invention device can be used for a variety of purposes, e.g., to identify the desired ECM conditions and/or compositions for further investigation of cell fate and behavior. Culturing employing the invention device can also be used for a variety of purposes, e.g., to grow the cells on a variety of ECM contained on the device to conduct a desired assay directly on the device.

In accordance with one embodiment of the present invention, the invention screening and culturing device has been used to study the effect of the composition of the extracellular matrix (including extracellular matrix proteins, adhesion molecules, small molecules, and the like) on paclitaxel induced apoptosis of the MCF-7 breast carcinoma cell line. 96 different extracellular matrix (ECM) combinations (composed of extracellular matrix proteins (ECMPs), signaling molecules and peptides, as well as various combinations thereof) were screened for differential cytotoxic profiles in MCF-7 cells cultured in the presence of paclitaxel.

It was surprisingly found that different ECM proteins, and particularly different combinations thereof, substantially alter the drug response. For example, MCF-7 cells in the presence of the combination of matrix components 80 μg/ml Fibronectin, Collagen IV and Collagen V, demonstrate cytotoxic resistance when compared with Fibronectin alone.

The invention screening and culturing device has, therefore, been demonstrated to be a novel and useful platform to facilitate understanding the interaction between tumor cells and the microenvironment in which they are found (e.g., cell-cell interactions, cell-matrix interactions, and the like) which in turn will be useful to improve the therapeutic effect of chemotherapeutic drugs.

In accordance with another embodiment of the present invention, the invention screening and culturing device has been used to study human BM MSC cell differentiation towards the cardiac lineage in the presence of 96 different extracellular matrix (ECM) combinations (composed of extracellular matrix proteins (ECMPs), signaling molecules and peptides, as well as various combinations thereof).

In accordance with one aspect of the present invention, it has been found that MSCs selectively attach to certain microenvironments. Some microenvironments support cell attachment better than others. It has also been found that selective extracellular matrix proteins alone or in various combinations support differentiation of MSCs towards cardiogenic lineage when stimulated by 5′-azacytidine, indicated by an increase in Nkx.2.5 expression. In contrast, cells exposed to other microenvironments exhibited no detectable changes in Nkx2.5 expression. Meanwhile, CD 29 (MSC expression marker) expression maintained unchanged overall after 5′-azacytidine treatment, regardless of the ECM conditions employed.

In accordance with yet another embodiment of the present invention, the invention screening and culturing device has been used to study the epithelial mesenchymal transition process for A549 lung cancer cell line in the presence of 96 different extracellular matrix (ECM) combinations (composed of extracellular matrix proteins (ECMPs), signaling molecules and peptides, as well as various combinations thereof).

In accordance with another embodiment of the present invention, it has been found that A549 cells demonstrate preferential attachment and distinct adherence morphologies to certain ECM combinations. This is an important finding. It illustrates the importance of cell substrate in cellular assay development. In accordance with one aspect of the present invention, it has been observed that certain ECM compositions promote cell attachment of A549 cells in the absence of TGF-β. However, cell detachment is observed upon addition of TGF-β, most likely due to the biological changes that occur in cells during EMT (FIG. 13). Because the invention screening and culturing device provides ECM spots which are substantially uniform in size and shape, the MicroMatrix™ system provides a consistent surface for cell adhesion, therefore improving consistency between experiments.

In accordance with yet another embodiment of the present invention, it has also been found that certain combinations of ECMs enhance TGF-β induced EMT. A549 cells attached to certain combinations of ECMs for 24 hrs in the presence of TGF-β demonstrated EMT related decreases in e-cadherin and increases in vimentin expression when compared to those from other ECM compositions. Conversely, some ECM compositions appeared to limit A549 cell transformation in the presence of TGF-β, further demonstrating the importance of cell-matrix interactions and its ability to dictate cellular fate and function in an in vitro setting.

### BRIEF DESCRIPTION OF THE FIGURES

In FIG. 1, each block, e.g. A1, is composed of 3-20 replicates of unique ECM conditions. The shaded area represents the frosted end of the glass slide.

FIG. 2 is an example of possible ECM conditions on the matrix screening and culturing device, wherein C1 is Collagen I; C3 is Collagen III; C4 is Collagen IV; C5 is Collagen V; C6 is Collagen VI; FN is Fibronectin; LN is Laminin; and VN is Vitronectin.

FIG. 3 provides a schematic diagram of the fabrication of a matrix screening and culturing device according to the present invention.

FIG. 4 presents the results of HUVEC cell attachment using the invention matrix screening and culturing device. ECM conditions are listed from top (best) to bottom (worst).

FIG. 5 presents the results of Jurkat cell attachment using the invention matrix screening and culturing device. ECM conditions are listed from top (best) to bottom (worst). Jurkat cells overall have limited ability to attach to the variety of ECMs.

FIG. 6 is a schematic of components printed on the invention matrix screening and culturing device, wherein C1 is Collagen I; C3 is Collagen III; C4 is Collagen IV; C5 is Collagen V; C6 is Collagen VI; FN is Fibronectin; LN is Laminin; VN is Vitronectin; and HA is Hyaluronic Acid.

FIG. 7 is an example of a 300 μm spot of Fibronectin with MCF-7 cells attached. The image was captured using Cellomics VTI at 20×.

FIG. 8 is a schematic of how the slide is divided up into multiple form factors encompassing multiple subarrays. The schematic includes 4 columns of subarrays for simplicity; the actual array had 6 columns of subarrays.

FIG. 9 represents the matrix components and geographical locations of components on the array to which between about 25 and about 200 cells attached.

FIG. 10 presents images of MCF-7 cells immobilized to fibronectin spots +/−exogenous paclitaxel, as captured by Cellomics VTI. Cells in the presence of paclitaxel are seen to undergo detachment from fibronectin, compromise of the cellular membrane, loss of mitochondrial membrane potential and relocalization of the mitochondria.

FIG. 11 collectively summarizes 4 parameters of cellular cytotoxicity: Cell Loss (see FIG. 11A), Cell Membrane Permeability (see FIG. 11B), Mitochondrial Trans-Membrane Potential (see FIG. 11C), and Cytochrome c loss (see FIG. 11D). Each of these parameters was quantified using the Cellomics VTI compartmental analysis bioapplication software. MCF-7 is seen to respond in a dose dependant manner to differing concentrations of paclitaxel in the presence of fibronectin.

FIG. 12 collectively summarizes 4 parameters of cellular cytoxicity of MCF-7 cells grown on Fibronectin alone vs. equal concentrations of Fibronectin, Collagen IV & Collagen V in combination upon exposure to paclitaxel. FIG. 12A relates to Cell Loss, FIG. 12B relates to Cell Membrane Permeability, FIG. 12C relates to Mitochondrial Trans-Membrane Potential, and FIG. 12D relates to Cytochrome c loss.

FIG. 13A illustrates that A549 cells show preferential attachment to different ECM compositions. FIGS. 13B and C present bright-field 20× images of A549 cells attached to ECM composition #11.

FIG. 14 collectively illustrate that A549 EMT is ECM composition dependent. FIGS. 14A, B, C and D are composite images of A549 cells attached to two different ECM compositions on the MicroMatrix™ ECM array (blue=nucleus, green=e-cadherin, red=vimentin). FIGS. 14A and B are images of A549 cells on ECM composition #11 which supports EMT in the presence of TGF-β. FIGS. 14C and D represent images of A549 cells on ECM composition #12 which did not support EMT in the presence of TGF-β. FIGS. 14E and F are graphs of corresponding quantitative data analyzed by Cellomics.

### DETAILED DESCRIPTION OF THE INVENTION

In accordance with the present invention, it has been determined that incorporating appropriate ECMs into any cell based assay can increase the validity of in vitro experiments and therefore, provide an accurate translation in vivo. For example, the utility of the invention screening and culturing device has been demonstrated for use in a method to identify matrices that enhance epithelial cancer cell transformation towards an invasive mesenchymal phenotype when stimulated by TGF-β. In a single assay, it is possible to monitor differential expression of EMT markers in A549 lung cancer epithelial cells adhered to different ECMs.

Therefore, in accordance with the present invention, a combinatorial ECM component screening and culturing device has been designed and manufactured. Such devices enable those of skill in the art to rapidly identify the appropriate individual components or combination of components that constitute ECMs for the optimal culturing of a particular cell type. Invention screening and culturing devices allow those of skill in the art to test up to about 10,000 ECM components or combinations of components using as little as about 20,000 cells. The invention screening and culturing device is applicable to all cell types with a potential for adherence to an ECM in a standard tissue culture vessel, including but not limited to embryonic and adult stem cells, primary cells, established cell lines (such as Jurkat T lymphoma, HEK, MG-63, HUVEC etc.) as well as cells isolated from fresh tissues.

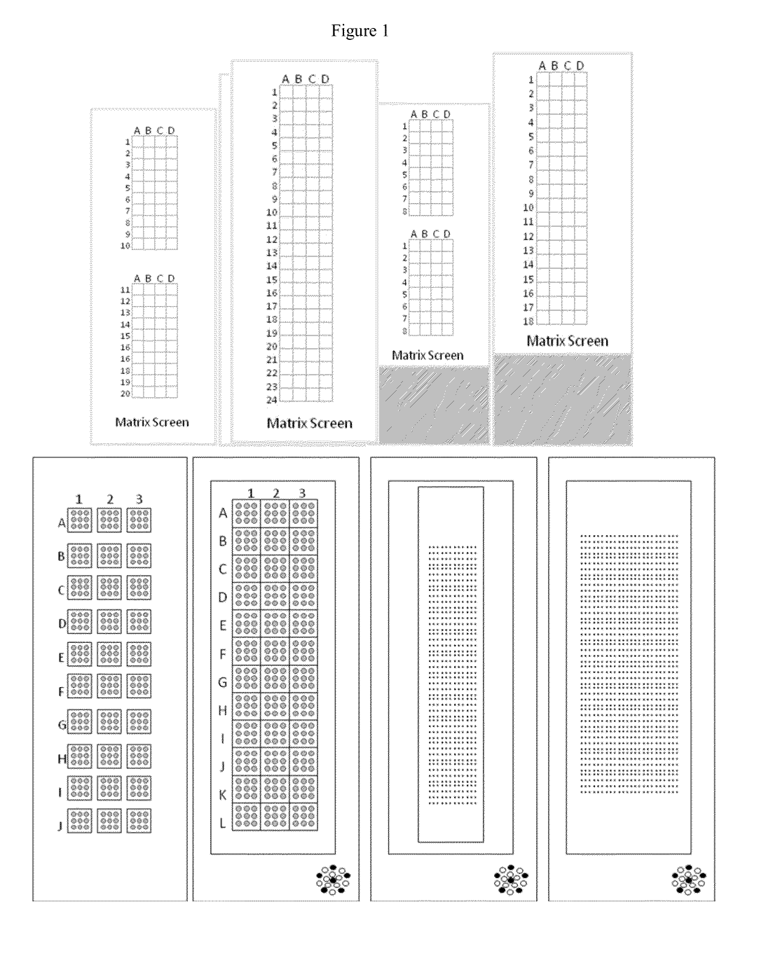

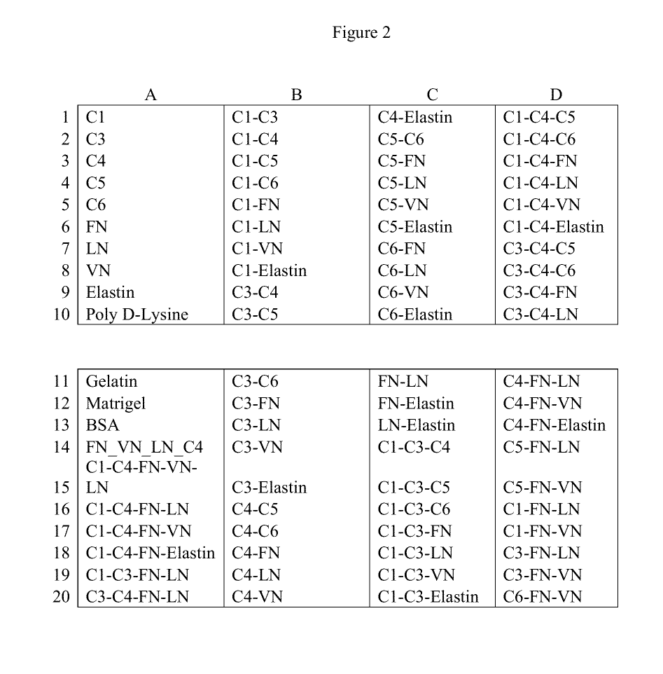

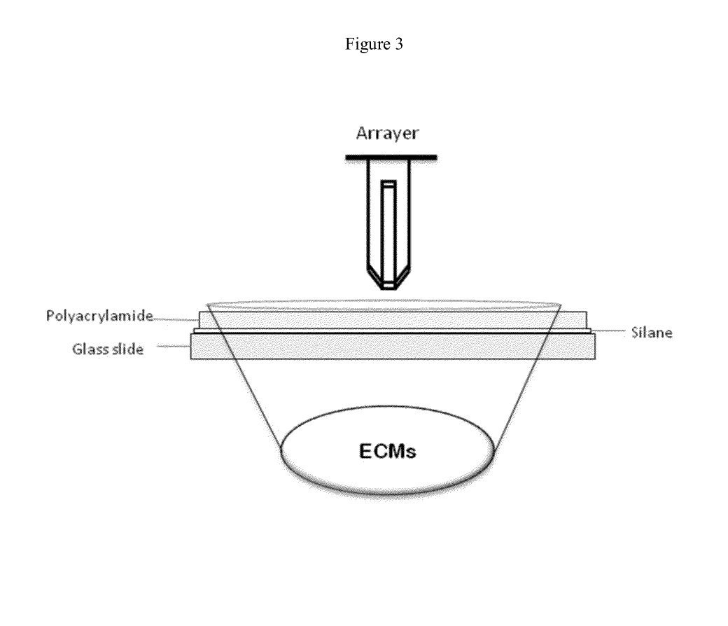

For example, a suitable support (e.g., a standard microscope slide (about 75 mm×25 mm×1 mm) or a plate (about 128 mm×86 mm×1.3 mm) made of glass or plastic (e.g., polystyrene, polypropylene, polycarbonate, and the like) can be coated with about a 20-100 μm thickness of hydrogel (e.g., polyacrylamide, agarose, silicone, methylcellulose, hyaluronan, and the like, or cross-linked biopolymers (e.g., polyvinylpyrrolidone, polyethylene oxide, and the like) gel) pad. Individual ECM components (concentration of about 0.01 mg/ml-1 mg/ml) as well as the combination of the components (total concentration of about 0.01 mg/ml-1 mg/ml) can be printed on the coated surface by an array printer, such as a MicroGrid printer (DigiLab, Holliston, Mass.). The resulting ECM spots are about 50-1000 μm in diameter. Each ECM is printed in replicates of about 3-20. The center to center distance of nearest spots is sufficient to substantially preclude overlap of the spots (typically the distance between spots is about 100 μm or more, preferably >about 150 μm). Markings on the glass slide indicate the rows and columns for easy identification of different ECM(s). See FIG. 1 for schematic diagrams of several representations of the array design. Each block on the slide is a group of replicates of a particular ECM. The examples of ECMs are listed in FIG. 2. The array may contain any combination of individual components.

In addition to ECM printed on the slides, fluorescent materials such as fluorescent dyes, fluorescent conjugated antibodies, fluorescence conjugated proteins, fluorescent conjugated polysaccharide, etc. may also be printed on the slides at one or more positions thereof to provide geographical location marks on the slides.

An ECM component screening and culturing device according to the present invention can be placed into a tissue culture vessel. In excess of about 20,000 cells suspended in culture media are then added onto the top of the slide. The cells are incubated in the presence of culturing media with the device for about 30 min up to about 30 days dependent on the cell type to allow cell attachment to appropriate ECMs. The optimal ECM conditions are then identified by observing the cell's morphology and quantity under phase contrast microscope. Cells on the device can also be imaged using any type of immunofluorescent stains, for example by nuclear staining such as PoPo-3 (Life Technology) or DraQ5 (Cell Signaling). Specific cell behavior markers can also be investigated by specific antibody staining. Images can be captured and quantitative data can be deciphered in a variety of ways, e.g., by using a fluorescence microscope, an array scanner, a high content imager, and the like.

An exemplary process for the manufacture of an ECM screening and culturing device according to the present invention is as follows. Glass slides (75 mm×25 mm×1 mm) are washed with a suitable organic solvent (e.g., about 30 min in acetone, then about 30 min in methanol, and the like), then 10 times in Millipore water (MQH₂O). The slides are then etched about 1 hour to overnight in 0.02-0.2 N NaOH, rinsed five times with MQH₂O, then dried in an oven (at 55° C.-85° C.) for 1 hour. The slides are then silanized for about 1 hr to overnight in a 2% solution of 3-(trimethoxysilyl)propyl methacrylate in anhydrous toluene, and dried for about 15 min to about 1 hour in an oven (at 55° C.-85° C.).

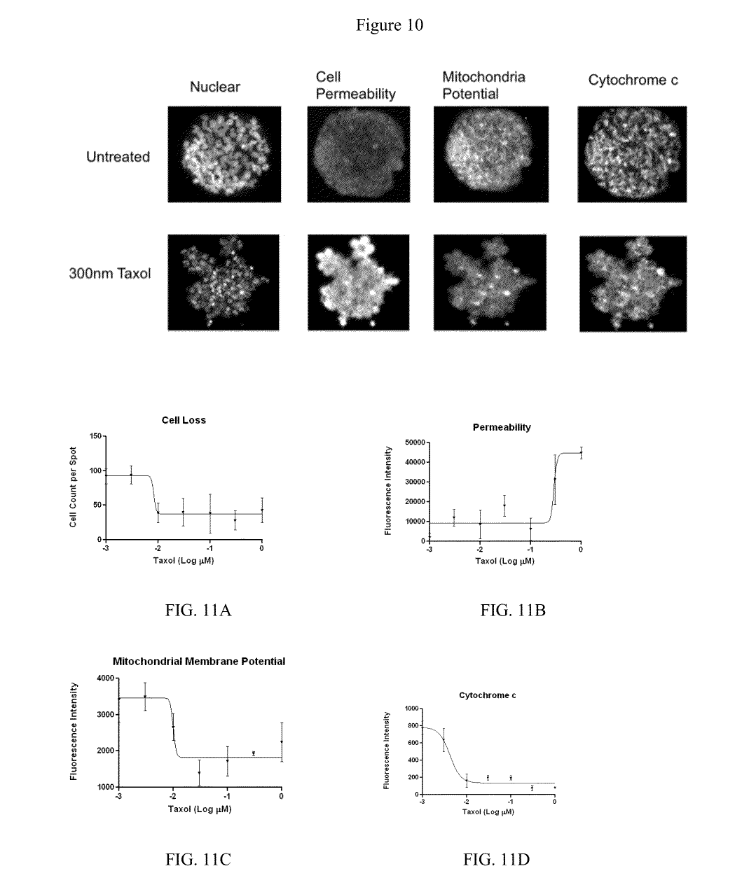

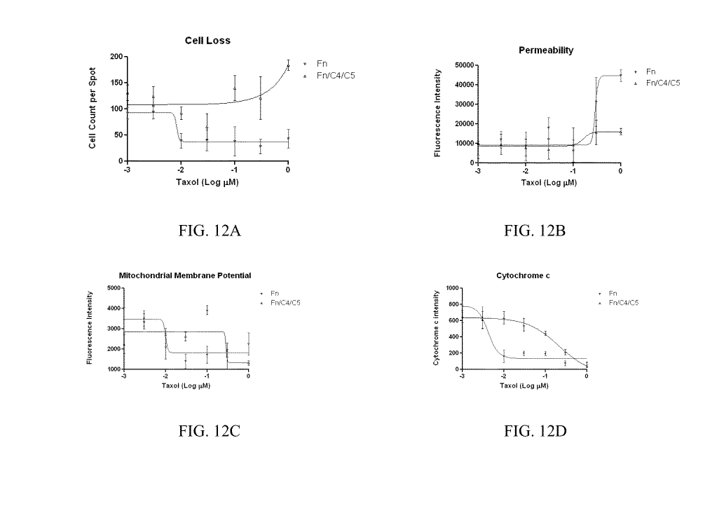

About 40-100 μL of solution comprising 8.0%-15.0% (w/v) acrylamide, 0.55% (w/v) bisacrylamide, and 10% (w/v) photoinitiator Irgacure 2959 (20 μg/mL-200 μg/mL) is placed on a silanized slide and covered with a 75 mm×25 mm or 60 mm×24 mm cover slip. The slide is then exposed to ultraviolet A light for 7-10 min and immersed in MQH₂O for about 2 min. The cover slip is then removed, leaving a thin (about 40-100 μm thickness) polyacrylamide gel pad. The polyacrylamide slides are soaked in MQH₂O overnight, and then dried on a hot plate (at about 60° C.) for about 10 min (see FIG. 10).

Stock solutions of ECM components (about 0.01 mg/ml-1 mg/ml), such as, but not limited to Collagen I, III, IV, V, VI, Fibronectin, Laminin, Vitronectin, Elastin, Tenascin, Decorin, etc. as well as, RGD peptide, Poly D/L-lysine, Matrigel™ (BD Biosciences), gelatin and BSA (negative control) are mixed 1:1 with 200 mM acetate, 10 mM EDTA, 40% (v/v) glycerol, and 0.5% (v/v) Triton X-100 in MQH₂O, at pH 4.9-8.5. Individual and combinations of different components are mixed in 384-well plates.

Printings can be performed using an arrayer such as MicroGrid or SpotArray 24 (Perkin Elmer, Waltham, Mass.) at room temperature with about 30-75% relative humidity according to instrument manufacturer's instructions. To control for variability, each spot is printed in replicates (about 3-20) as a block of subarray. Array slides are dehydrated and frozen at −20° C. Array slides are shipped on ice packs. Upon receipt, an array slide can be stored at about 65-75% humidity (in the presence of saturated NaCl solution) at 4° C. or frozen at −20° C. for a minimum of about six months.

As an example of the usage of the invention screening and culturing device, prior to their use, slides are placed in a sterile container, such as Nunc 4 well rectangle culture plate (Thermo Fisher) and washed once with PBS.

Thereafter, a cell type for which one would like to identify an appropriate ECM (e.g., embryonic and adult stem cells, cardiac cells, neural cells, lymphocytes, hepatocytes, and the like) is suspended in cell culture medium and added on the ECM component screening and culturing device (about 0.2-20×10⁵ cells per slide) and allowed to settle on the ECM component derived spots for >15 min to days (dependent on the cell type) prior to rinsing with the medium to remove unattached cells and debris. Due to the non-fouling nature of the acrylamide gel pad on the slides, cells are confined to the printed spots. The device in the culturing vessel can be placed under phase contrast microscope to observe the quantity and morphology of the cells at each of the ECM conditions. With the guidance of the marking on the slide, one can readily identify the ECM components on the slide. If long term culturing is required, cell media can be replenished as needed.

Alternatively, cells attached on the invention screening and culturing device can be fixed and stained on the device. The arrays are then washed three times with a suitable solution (such as HBSS or PBS), then fixed in 4% paraformaldehyde (PFA) made in PBS for 5 min at 4° C., followed by 10 min at room temperature, or using other standard cell fixation techniques such as ice cold methanol. Quantitative data can be obtained through image analysis of immunofluorescent staining. Cells can be stained by nuclear stains, such as POPO-3 or DraQ5, or any other immunofluorescent method including specific antibodies or in-situ mRNA. The resulting device is then air-dried and can be imaged in a variety of ways, e.g., on a fluorescence microscope, an array scanner, a high content imager, or the like. Cells on the device can also be stained for specific cell markers to investigate certain cell behaviors. Cells are permeabilized with 0.2% (v/v) Triton X-100 and blocked with 1% (w/v) BSA and 3% (w/v) milk for about 30 min. Cells on the slides are then stained with antibodies against cell markers for about 1 hour to overnight, then washed three times with TBS or PBS, and incubated with suitable secondary antibody for about 1 hour. The resulting device is then air-dried and can be imaged in a variety of ways, e.g., on a fluorescence microscope, a high content imaging system, an array scanner, a high content imager, or the like. Images generated from an array scanner can be quantified using a commercial program, such as GenePix software (MDS Analytical Technologies, Sunnyvale, Calif.), or any other high content imaging or standard fluorescent image analysis software.

The examples below relate to cell studies using the invention matrix screening and culturing device. All examples were carried out using an ECM component screening and culturing device containing individual and combinations of the following components: Collagen I (human, rat, bovine), Collagen IV (human), Vitronectin (human), Tropo-Elastin (human), Poly-D-lysine, RGD peptide (all from Advanced BioMatrix), Laminin (Sigma), gelatin (Sigma) and Matrigel™ (BD Bioscience).

Polyacrylamide gel coated slides are prepared as described above. A set of SMP 4.0 pins were used for printing the ECMs using an arrayer MicroGrid. Each spot is about 300 μm in diameter with about a 550 μm spot center-to-center distance in replicates of nine. Total of 60 ECM conditions were printed.

Complex microenvironment(s) contemplated for use in the practice of the present invention comprise two or more components selected from the group consisting of extracellular matrix proteins or components thereof, cellular adhesion molecules, monosaccharides, oligosaccharides, polysaccharides, glycoproteins, proteoglycans, non-proteoglycan polysaccharides, cell communication molecules, complex carbohydrates, lipids, vitamins and metabolites thereof, naturally occurring low molecular weight biologically active molecules, synthetic low molecular weight biologically active molecules, polypeptides, synthetic polymers, biopolymers, antibodies, nucleic acids, inorganic salts, media supplements, and the like.

Each of the microenvironments contemplated for use herein comprise a multi-factorial array of at least two or more components selected from the group consisting of extracellular matrix proteins or components thereof, cellular adhesion molecules, monosaccharides, oligosaccharides, polysaccharides, glycoproteins, proteoglycans, non-proteoglycan polysaccharides, cell communication molecules, complex carbohydrates, lipids, vitamins and metabolites thereof, naturally occurring low molecular weight biologically active molecules, synthetic low molecular weight biologically active molecules, synthetic polymers, biopolymers, antibodies, nucleic acids, inorganic salts, media supplements, and the like. As readily recognized by those of skill in the art, biologically active components may fit into more than one of the categories set forth above, e.g., growth factors may also be considered to be signaling molecules.

In certain aspects of the invention, each of the microenvironments contemplated for use herein comprise a multi-factorial array of three or more components selected from the various components set forth above.

In certain aspects of the invention, each of the microenvironments contemplated for use herein comprise a multi-factorial array of four or more components selected from the various components set forth above.

In certain aspects of the invention, each of the microenvironments contemplated for use herein comprise a multi-factorial array of five or more components selected from the various components set forth above.

In certain aspects of the invention, each of the microenvironments contemplated for use herein comprise a multi-factorial array of six or more components selected from the various components set forth above.

In certain aspects of the invention, each of the microenvironments contemplated for use herein comprise a multi-factorial array of seven or more components selected from the various components set forth above.

In certain aspects of the invention, each of the microenvironments contemplated for use herein comprise a multi-factorial array of eight or more components selected from the various components set forth above.

In certain aspects of the invention, each of the microenvironments contemplated for use herein comprise a multi-factorial array of nine or more components selected from the various components set forth above.

In certain aspects of the invention, each of the microenvironments contemplated for use herein comprise a multi-factorial array of ten or more components selected from the various components set forth above.

Thus, any one micro-environment will likely contain less than all of the various components referred to above, but at least one representative component referred to above will be represented in at least one of the micro-environments of a given multi-factorial array.

As readily recognized by those of skill in the art, the number of components combined to create a given microenvironment can be widely varied, as can the relative amounts of the various components contemplated for use herein to prepare the multi-factorial array. An exemplary population of microenvironments can be generated by creating various optional combinations of the components contemplated for use herein, as illustratively set forth in the following table, wherein “+” indicates a component is present (and ++ or +++ indicate the presence of a higher relative amount of such component, relative to a component which is merely “present”); and “−” indicates a component is not present in the particular microenvironment).

### Microenvironment

| Component | 1 | 2 | 3 | 4 | 5 | 6 | 7 | 8 | 9 | 10 |
|---|---|---|---|---|---|---|---|---|---|---|
| Extracellular matrix | +++ | +++ | +++ | ++ | ++ | ++ | + | + | + | − |
| proteins | | | | | | | | | | |
| Cellular adhesion | − | + | + | + | ++ | ++ | ++ | +++ | +++ | +++ |
| molecules | | | | | | | | | | |
| Saccharides | + | − | + | − | + | − | + | − | + | − |
| Glycoproteins | − | + | − | + | − | + | − | + | − | + |
| Proteoglycans | + | − | + | − | + | − | + | − | + | − |
| Cell communication | − | + | − | + | − | + | − | + | − | + |
| molecules | | | | | | | | | | |
| Complex | + | − | + | − | + | − | + | − | + | − |
| carbohydrates | | | | | | | | | | |
| Lipids | − | + | − | + | − | + | − | + | − | + |
| Vitamins | + | − | + | − | + | − | + | − | + | − |
| Biopolymers | − | + | − | + | − | + | − | + | − | + |
| Antibodies | + | − | + | − | + | − | + | − | + | − |
| Nucleic acids | − | + | − | + | − | + | − | + | − | + |
| Inorganic salts | + | − | + | − | + | − | + | − | + | − |
| Media supplements | − | + | − | + | − | + | − | + | − | + |

As used herein, the term “extracellular matrix proteins” refers to structural proteins which provide structural integrity to cells. Exemplary extracellular matrix proteins contemplated for use herein, or functional components thereof, include collagen, fibronectin, laminin, elastin, vitronectin, tenascin, decorin, and the like, as well as combinations of any two or more thereof.

Exemplary collagens contemplated for use herein include Type I fibrillar collagen, Type II fibrillar collagen, Type III fibrillar collagen, Type V fibrillar collagen, Type XI fibrillar collagen, Type IX facit collagen, Type XII facit collagen, Type XIV facit collagen, Type VIII short chain collagen, Type X short chain collagen, Type IV basement membrane collagen, Type VI collagen, Type VII collagen, Type XIII collagen, and the like, as well as combinations of any two or more thereof.

Exemplary cellular adhesion molecules (CAM) contemplated for use herein, or components thereof, include members of the immunoglobulin superfamily (IgSF CAMs), the integrins, the cadherins, the selectins, the lymphocyte homing receptors, and the like, as well as combinations of any two or more thereof.

Exemplary mono- and oligosaccharides contemplated for use herein, or components thereof, include trioses, tetroses, pentoses, hexoses, heptoses, octoses, nonoses, sucrose, lactose, maltose, trehalose, turanose, cellobiose, raffinose, melezitose, malotriose, acarbose, stachyose, and the like, as well as combinations of any two or more thereof.

Exemplary glycoproteins contemplated for use herein, or components thereof, include proteoglycans and non-proteoglycan polysaccharides, and the like, as well as combinations of any two or more thereof. Exemplary glycoproteins include heparin, heparan sulfate, chondroitin sulfate, dermatan sulfate, keratan sulfate, hyaluronic acid, perlecan, aggrecan, versican, decorin, biglycan, fibromodulin, lumican, and the like, as well as combinations of any two or more thereof.

Cell communication molecules contemplated for use in the practice of the present invention include growth factors, hormones, signaling molecules, cytokines, and the like, as well as combinations of any two or more thereof.

Growth factors contemplated for use herein include any substance capable of stimulating cellular growth, proliferation and/or differentiation, typically a protein or a steroid hormone. Presently preferred growth factors are endogenous to the species of organism from which the desired cell population is obtained, or endogenous to a species homologous to the species from which the desired cell population is obtained, as well as combinations of any two or more thereof. Growth factors are sometimes referred to in the art as cytokines, although as used herein, cytokines are but a subset of the compounds contemplated for use herein.

Exemplary growth factors include angiopoietin-1, angiopoietin-2, brain-derived neurotrophic factor (BDNF), one or more members of the BMP signaling family, one or more members of the Wnt family, osteopontin, one or more members of the epidermal growth factor (EGF) family, one or more members of the epidermal growth factor-CriptoFRL1Cryptic (EGF-CFC) family, EPO, Eotaxin, one or more members of the fibroblast growth factor (FGF) family, FLT-3 ligand, one or more members of the hepatocyte growth factor (HGF) family, one or more members of the insulin growth factor (IGF) family, platelet-derived growth factor, sonic hedgehog, one or more members of the transforming growth factor (TGF), family, TPO, one or more members of the vascular endothelial growth factor (VEGF) family, PIGF, Rantes, stromal cell-derived factor (SDF), Granulocyte-colony stimulating factor (G-CSF), granulocyte-macrophage colony stimulating factor (GM-CSF), growth differentiation factor-9 (GDF9), hepatoma derived growth factor (HDGF), myostatin (GDF-8), neurotrophins such as nerve growth factor (NGF), platelet-derived growth factor (PDGF), amine-derived hormones, peptide hormones, lipid and phospholipid-derived hormones, the IL-2 subfamily, the interferon (IFN) subfamily, the IL-10 subfamily, the IL-1 family, the IL-17 family, and the like, as well as combinations of any two or more thereof.

Exemplary hormones contemplated for use herein include steroids, retinoic acid, thyroid hormone, vitamin D3, insulin, parathyroid hormone, luteinizing hormone releasing factor (LHRH), alpha and beta seminal inhibins, human growth hormone, and the like.

Exemplary cytokines contemplated for use herein include GM-CSF, G-CSF, M-CSF, one or more members of the interferon family, one or more members of the interleukin family, one or more members of the TNF family, one or more members of the transferrin family, insulin, one or more members of the human growth hormone (HGH) family, one or more prostanoids, one or more members of the prostaglandin hormone family, GRO/KC/CINC chemokines, kallikrein, oncostatin, osteoprotegerin, one or more members of the sphingosine family, one or more MCP/MCAF chemokines, MIG, macrophage inflammatory protein (MIP) chemokines, and the like, as well as combinations of any two or more thereof.

Exemplary signaling components contemplated for use herein include any signaling component endogenous to the species of organism from which the cell population is obtained, or a species homologous to the species from which said cell population(s) were obtained, as well as combinations of any two or more thereof. Such signaling molecules include GPCR, activin, BMP, neurotrophic factors, and the like, as well as combinations of any two or more thereof.

Exemplary complex carbohydrates contemplated for use herein include calcium-independent IgSF CAMs (such as, for example, immunoglobulin superfamily CAMs (IgSF CAMs) including homophilic or heterophilic species which bind integrins or different IgSF CAMs; examples of some members of this family include neural cell adhesion molecules (NCAMs), intercellular cell adhesion molecule (ICAM-1), vascular cell adhesion molecule (VCAM-1), Platelet-endothelial Cell Adhesion Molecule (PECAM-1), L1, CHL1, MAG, nectins and nectin-like molecules, and the like); integrins (a family of heterophilic CAMs that bind IgSF CAMs or the extracellular matrix), lymphocyte homing receptors (also known as addressins, includingCD34 and GLYCAM-1), and the like); calcium-dependent IgSF CAMs (such as, for example, cadherins (a family of homophilic CAMs, Ca²⁺-dependent, such as E-cadherins (epithelial), P-cadherins (placental), and N-cadherins (neural), selectins (a family of heterophilic CAMs that bind fucosylated carbohydrates, e.g., mucins, including E-selectin, (endothelial), L-selectin (leukocyte), and P-selectin (platelet), and the like, as well as combinations of any two or more thereof.

Exemplary naturally occurring low molecular weight biologically active molecules contemplated for use herein include hormones, retinoic acid, ICCB Known Bioactives Library (BioMol) (see, for example, Pan, H. et. al., J. Biol. Chem. 2008 283 33808; Zhang, L. et. al., PNAS 2007 104 19023; and A. Shelat and K. G. Guy, Nat. Chem. Biol. 2007 3 442); Natural Products Library (BioMol) (see, for example, M. Tulp et al. Drug Discov. Today 2004 9 450; A. Harvey Drug Discov. Today 2000 5 294; D. J. Newman et al. J. Nat. Prod. 2003 66 1022; and M. Heinrich et al. J. Pharm. Pharmacol. 2001 53 425), and the like, as well as combinations of any two or more thereof.

Exemplary synthetic low molecular weight biologically active molecules contemplated for use herein include MaxiVerse™ from Molecular Diversity Libraries (MolBio), LOPAC¹²⁸⁰ (from Sigma), MyriaScreen Diversity Collection of drug-like screening compounds (from Sigma), compound libraries available on the world-wide web from biofocus.com/offerings/compound-libraries.htm?gclid=CMXYzorejp4CFSZdagodhktmsw, and the like, as well as combinations of any two or more thereof.

Additional exemplary naturally occurring and synthetic low molecular weight biologically active molecules contemplated for use herein include antiproliferatives, enzyme inhibitors, cell cycle regulators, apoptosis inducers, GPCR ligands, second messenger modulators, nuclear receptor ligands, actin and tubulin modulators, kinase inhibitors, protease inhibitors, ion channel blockers, gene regulation agents, lipid biosynthesis inhibitors, phosphodiesterase inhibitors, G-Proteins, cyclic nucleotides, multi-drug resistance, neurotransmission inhibitors, phosphatase inhibitors, and the like, as well as combinations of any two or more thereof.

Exemplary polypeptides contemplated for use herein include protein transduction domain (PTD) peptides, and the like, as well as combinations of any two or more thereof.

Exemplary biopolymers contemplated for use herein include polyalkylene oxides, poly(ethylene glycol-co-acryloyl glycolic caproic acid), poly(acryloyl-6-amino caproic acid), poly(acryloyl-2-acrylamido glycolic acid), poly(2-hydroxyethyl methacrylate), poly(N-isopropylacylamide), poly(trimethylene carbonate), poly(acryloyl-4-aminobenzoic acid), poly(acrylamido-methyl-propane sulfonate), poly(3-(methacryloylamino)propyl)dimethyl(3-sulfopropyl)ammonium hydroxide), poly(3-(methacryloylamino)propyl)timethylammonium chloride, poly(ethylene-co-acrylic acid), poly(acrylic acid), poly(L-lactide), poly(D-lactide), poly(DL-lactide-co-glycolide) 85:15, poly(DL-lactide-co-glycolide) 75:25, poly(DL-lactide-co-glycolide) 65:35, poly(DL-lactide-co-caprolactone) 86:14, poly(DL-lactide-co-caprolactone) 40:60, polycaprolactone, poly(3-hydroxybutyric acid-co-3-hydroxyvaleric acid), poly(3-hydroxybutyric acid), poly(propylene carbonate), poly(methyl vinyl ether-alt-maleic anhydride), hydrophilic, poly(sodium 4-styrenesulfonate), poly-L-arginine hydrochloride, poly-D-lysine hydrobromide, poly-L-glutamic acid sodium salt, poly-L-ornithine hydrobromide, poly(2-ethyl-2-oxazoline), poly(oligoethylene glycol methyl ethyl methacrylate), poly(butyl methacrylate), poly(ethyl methacrylate), poly(styrene-co-methacrylic acid), poly-L-arginine hydrochloride, poly(ethylene glycol)methacrylate, poly(styrene-alt-maleic acid), poly(styrene), poly(ethylene-alt-maleic anhydride), poly(4-styrenesulfonic acid-co-maleic acid), poly(methyl vinyl ether-alt-maleic acid), poly(methyl vinyl ether), poly(styrene-co-maleic anhydride), poly(isobutylene-co-maleic acid), poly(maleic anhydride-alt-1-octadecene), poly(styrene-alt-maleic anhydride), partial methyl ester, poly(ter-butyl methacrylate), poly(2-hydroxyethyl methacrylate), poly(benzyl methacrylate), poly(2-(dimethylamino)ethyl methacrylate), poly(4-vinylphenol-co-methyl methacrylate), poly(ethylene-co-gylcidyl-methacrylate), poly(cyclohexyl methacrylate), poly(tert-butyl acrylate-co-ethyl acrylate-co-methacryalic acid), poly(ethylene-co-methyl acrylate-co-glycidyl methacrylate), poly(ethylene-co-acrylic acid), poly(ethylene-co-acrylic acid), poly(vinyl alcohol), poly(vinylphosphonic acid), poly(vinyl sulfate) potassium salt, poly(4-vinylpyridine hydrochloride), poly(4-vinylphenol), poly(4-vinylpyridine) crosslinked, poly(vinyl-co-ethylene), poly(vinyl butyral-co-vinyl alcohol-co-vinyl acetate), poly(ethylene-co-vinyl acetate-co-carbon monoxide), poly(allylamine hydrochloride), poly(anetholesulfonic acid), poly(epoxysuccinic acid), poly(1,4-butylene terephthalate), poly(styrene-co-4-bromostyrene-co-divinylbenzene), poly(1,6-hexanediol/neopentyl glycol-alt-adipic acid), poly(acrylonitrile), poly(styrene-co-allyl alcohol), poly(N′,N′-(1,3-phenylene)-isophthalamide), poly(trimellitic anhydride chloride-co-4′,4′-methylene-dianiline), poly(Bisphenol A carbonate), poly(azelaic anhydride), poly(trimethylolpropane di(propylene glycol)-alt-adipic acid/phthalic anhydride), poly(di(ethylene glycol adipate), poly(allyamine), poly(diallyl dimethyl ammonium), poly(diallyl methylamine hydrochloride), poly(1-glyceryl monomethacrylate), poly(3-chroloro-2-hydroxypropyl-2-methacroxyethydimethylammonium chloride), poly(butadienne maleic acid), poly(vinyl pyrroliodone), poly(n-vinylpyrrolidone-vinylacetate), poly(ethylenimine), chitosan, poly(1-glyceryl monomethacrylate), and the like, as well as combinations of any two or more thereof.

Exemplary nucleic acids contemplated for use herein include oligonucleotides, DNA molecules, RNA molecules, and the like, as well as combinations of any two or more thereof.

Exemplary DNA molecules contemplated for use herein include DNA-plasmids/vectors encoding Zinc-finger nucleases, Zinc-finger transcription factors, cDNA over-expression libraries, and the like, as well as combinations of any two or more thereof.

Exemplary RNA molecules contemplated for use herein include siRNA (see, for example, sigmaaldrich.com/life-science/functional-genomics-and-rnai/sirna.html on the world-wide web), shRNA (see, for example, (sigmaaldrich.com/life-science/functional-genomics-and-rnai.html and openbiosystems.com/RNAi/shrnaLibraries/ as available on the world-wide web), microRNA (see, for example, mirbase.org/index.shtml as available on the world-wide web), and the like, as well as combinations of any two or more thereof. As readily recognized by those of skill in the art, RNA molecules can be spotted onto a support comprising a plurality of complex microenvironments thereon either directly (e.g., using siRNA or microRNA), or as a virus containing a viral expression vector containing the RNA molecule of interest (e.g., microRNA or shRNA).

Exemplary lipids contemplated for use herein, or components thereof, include fatty acyls, glycerolipids, glycerophospholipids, sphingolipids, sterol lipids, prenol lipids, saccharolipids, polyketides, and the like, as well as combinations of any two or more thereof.

Exemplary vitamins and metabolites thereof (e.g., retinoic acid is a metabolite of Vitamin A), or functional components thereof, include vitamin A, vitamin B, vitamin C, vitamin D, vitamin E, vitamin K, and the like, as well as combinations of any two or more thereof.

Exemplary inorganic salts contemplated for use herein, or functional components thereof, include calcium chloride (CaCl₂), ferric nitrate (Fe(NO₃), magnesium sulfate (MgSO₄), potassium chloride (KCl), sodium bicarbonate (NaHCO₃), sodium chloride (NaCl), sodium phosphate dibasic (Na₂HPO₄—H₂O), cupric sulfate, manganese chloride, sodium selenite, zinc sulfate (ZnSO₄-7H₂O), sodium phosphate monobasic (NaH₂PO₄—H₂O), magnesium chloride (anhydrous), ferric sulfate (FeSO₄-7H₂O), and the like, as well as combinations of any two or more thereof.

Exemplary media supplements contemplated for use herein, or functional components thereof, include linoleic acid, lipoic Acid, hypoxanthine Na, putrescine 2HCl, sodium pyruvate, thymidine, knock-out serum replacement, glutamine and derivatives thereof, human plasmanate, and the like, as well as combinations of any two or more thereof.

As used herein, reference to a “plurality of complex microenvironments” embraces the use of array technology wherein a substantial number of microenvironments are applied to a single substrate. Typically, in the range of about 100 up to about 250,000 different microarrays are applied to a single substrate. An advantage of the methodology contemplated for use herein is the ability to screen a multitude of possible media formulations with a relatively small number of test cells, typically only about 250,000 cells are required to seed a support comprising a plurality of complex microenvironments thereon, said support typically comprising in the range of about 100 up to about 250,000 different microenvironments (depending on the seeding level employed (wherein arrays contemplated for use herein comprise anywhere from a single cell per well/spot, up to about 500 cells (or more) per well/spot)). Substrates contemplated for use herein preferably comprise in the range of about 10 up to about 200,000 different microenvironments, with substrates comprising in the range of about 20 up to about 100,000 microenvironments being presently preferred.

In accordance with the present invention, a plurality of complex microenvironments, wherein each complex microenvironment comprises a plurality of the above-described components, are created employing techniques which are well known in the art. For example, glass slides can be cleaned, silanized, and then functionalized with a gel coating (e.g., acrylamide—which is presently preferred because of the non-fouling nature thereof, which facilitates confining test cells to the printed spots on the substrate). Various components contemplated for use in the plurality of microenvironments can then be applied individually or combinatorially to the slides employing techniques which are known in the art, e.g., a commercial arrayer.

The invention will now be described in greater detail with reference to the following non-limiting examples.

### EXAMPLES

### Example 1

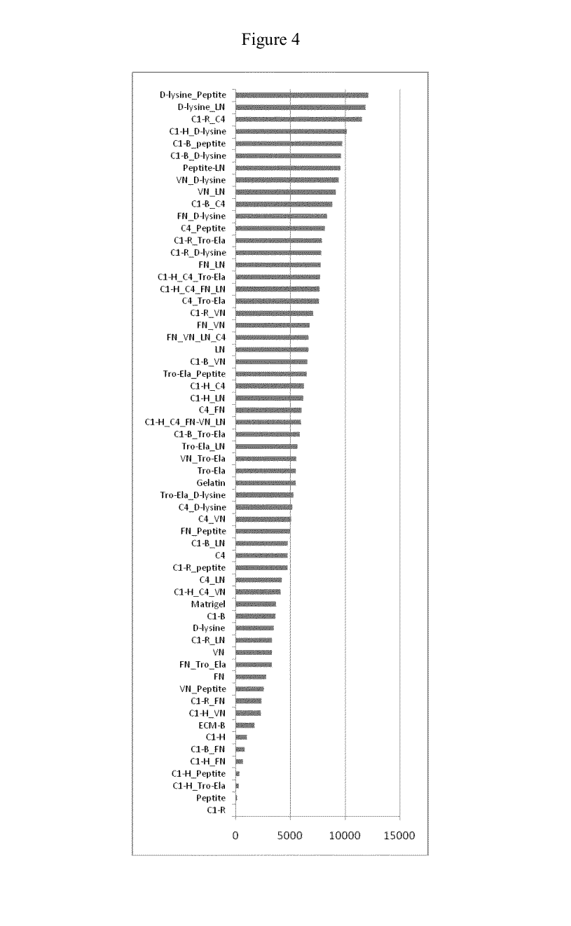

HUVEC cells in DMEM media containing 10% FBS were trypsinized from a T75 flask. Cells were resuspended at 50,000 cells/ml in DMEM media containing 10% FBS. 5 ml of cell suspension (total 250,000 cells) was added to the device that was placed in a 4 well NUNC cell culture vessel. The cells were allowed to attach for 18 hours in an incubator at 37° C. After 18 hours the vessel containing the device was removed from 37° C. Cells on the device were observed under a phase contrast microscope. Media was aspirated from the vessel containing the device. The resulting devices were then washed with HBSS twice and 4% paraformaldehyde (PFA) made in PBS was added to the vessel containing the device for 5 min at 4° C., followed by 10 min at room temperature. Paraformaldehyde was aspirated and the device was washed with HBSS. DraQ5 (Cell Signaling) made in PBS was added to the vessel containing the device for 5 minutes. DraQ5 solution was then aspirated and washed with PBS (5 mls) three times. The device was then removed from the vessel and air dried for 3 hours. The slide was then imaged using an array scanner Axon 4000B. Images were analyzed using a GenePix software (MDS Analytical Technologies, Sunnyvale, Calif.). Data are presented in FIG. 4.

### Example 2

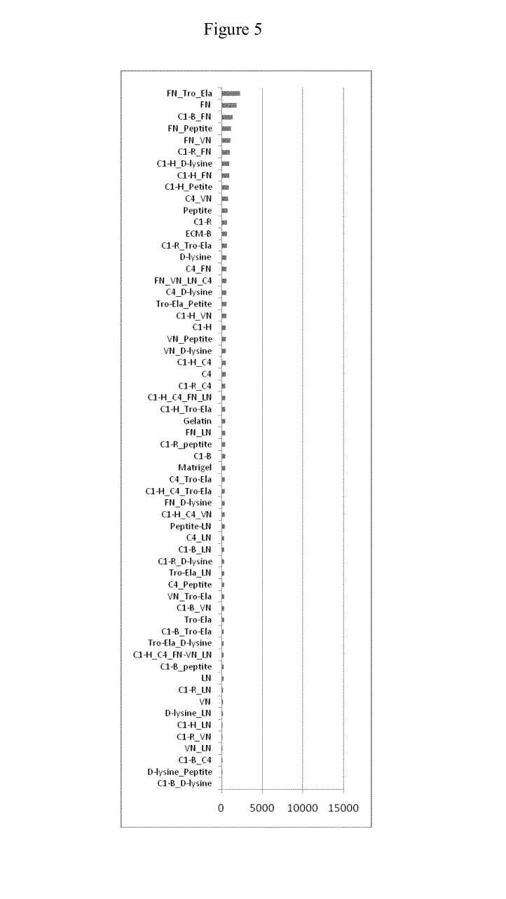

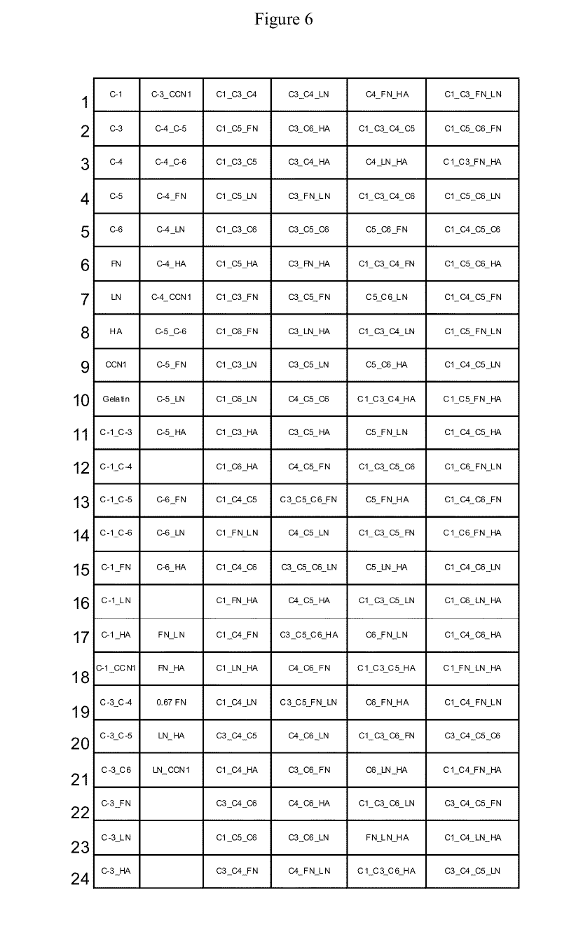

Jurkat cells in RPMI media containing 10% FBS were grown as suspension culture in a T75 flask. 5 ml of cell suspension (total 250,000 cells) was added to the device that was placed in a 4 well NUNC cell culture vessel. The cells were allowed to attach for 18 hours in an incubator at 37° C. After 18 hours the vessel containing the device was removed from 37° C. Cells on the device were observed under a phase contrast microscope. Media was aspirated from the vessel containing the device. The resulting devices were washed with HBSS twice and 4% paraformaldehyde (PFA) made in PBS was added to the vessel containing the device for 5 min at 4° C., followed by 10 min at room temperature. Paraformaldehyde was aspirated and the device was washed with HBSS. DraQ5 (Cell Signaling) made in PBS was added to the vessel contain the device for 5 minutes. DraQ5 solution was then aspirated and washed with PBS (5 mls) three times. The device was then removed from the vessel and air dried for 3 hours. The slide was then imaged using an array scanner Axon 4000B. Images were analyzed using a GenePix software (MDS Analytical Technologies, Sunnyvale, Calif.). Data are presented in FIG. 5.

### Example 3

### Use of Invention Device and Methods to Identify Extra Cellular Matrix Protein Combinations that Promote Paclitaxel Resistance in MCF-7 Breast Carcinoma Cells

Prior to use, slides are placed in a sterile container, such as a 4-chambered Nunc rectangle culture plate (Thermo Fisher) and soaked in PBS while being exposed to UVC germicidal radiation in a sterile flow hood for a minimum of about 30 min.

The human breast adenocarcinoma cell line MCF-7 was purchased from American Type Culture Collection (ATCC). Cells were cultured in minimal essential medium (DMEM), supplemented with 10% FBS and antibiotics (100 IU/ml of penicillin G), then incubated at 37° C. in a humidified atmosphere of 5% CO₂. Fetal bovine serum (FBS), DMEM, penicillin G and streptomycin were purchased from GIBCO/BRL-Invitrogen (Carlsbad, Calif., USA).

Paclitaxel (Taxol) was obtained from Sigma (St. Louis, Mo.). Cellomics Multiparameter Cytotoxicity 3 kit and Nunc Rectangular 4 well plates were purchased from Fisher Scientific. Formaldehyde (16%) was purchased from Thermo Scientific.

MCF-7 cells were seeded into 4 well plates containing an exemplary microarray slide according to the present invention at a density of about 0.1×10⁶ cells/ml in 5 ml complete medium (0.5×10⁶ cells/slide, total 8 slides) and cultured at 37° C. After 7 h of incubation, non adherent cells were removed and the slide was washed 1× with complete culture medium. New media with varying concentrations of paclitaxel was added ranging from 0 nM-3 uM (0 nM, 3 nM, 10 nM, 30 nM, 100 nM, 300 nm, 1 μM, 3 μM). Cells were incubated for about 16 h at 37° C.

Slides were processed using the protocol provided by the manufacturer of the Cellomics Multiparameter Cytotoxicity 3 kit, employing the reagents included in the kit. Briefly, 2 ml of media from each well of the 4 well plate was removed and 500 μl of live staining solution (complete media with Permeability Dye and Mitochondrial Membrane Potential Dye) was added into each well containing one individual microarray slide according to the present invention. The cells were incubated 37° C. for 30 min. Then media was removed and cells were fixed with 4% PFA for 20 min at room temperature. Slides were washed 1× with wash buffer and permeabilized with permeabilizing buffer for about 20 min at room temperature, protected from light. The slides were then washed 2 times with wash buffer and blocked with blocking buffer for about 15 min at room temperature. Blocking buffer was aspirated and the cells were incubated with 1 ml of Cytochrome c Primary Antibody in 1× Blocking buffer for about 1 h at room temperature. Cells were then washed 3 times with wash buffer and incubated with Secondary antibody (DyLight 649 Goat Anti-Mouse and Hoechst Dye in 1× Blocking Buffer) for about 1 h protected from the light. Cells were then washed one time with wash buffer and air dried for about 3 h at room temperature. The resulting slides were kept protected from light and analyzed within about 24 h.

The approximate absorption/emission maxima of the fluorescent dyes are as follows:
DyLight 649 Conjugates=646/674 nm
Mitochondrial Membrane Potential Dye=552/576 nm
Permeability Dye 491/509 nm
Hoechst Dye=350/461 nm

After processing with Cytotoxicity 3 Hit Kit, slides were imaged on the Cellomics VTI using the compartmental analysis Bioapplication software.

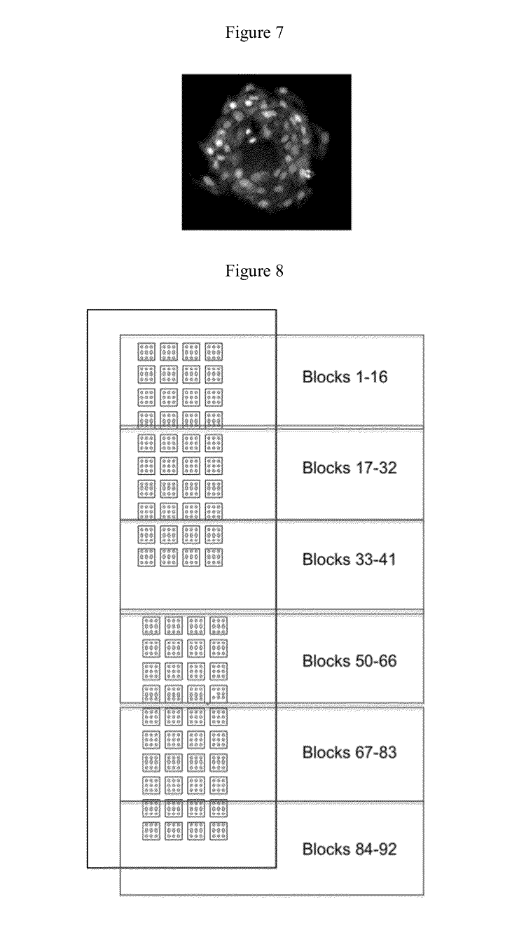

Briefly, 4 invention slides at a time were loaded into a ThermoFisher Slideport™ The Slideport™ was loaded onto the Cellomics VTI. Using the Cellomics Calibration Wizard, multiple custom form factors were designed to capture images of the roughly 1100 spots printed on each array. At 20× magnification, one complete 300-400 μm spot can be imaged without interference from other spots (see FIG. 7).

Scanning of multiple form factors was automated with the Cellomics plate ID wizard software which is included in the standard CellomicsSCAN software package. Sample spots were imaged and an exposure time was set for each channel. The exposure time was the same for each slide. After an entire slideport containing 4 slides was imaged, another slideport was loaded by hand. To ensure proper imaging of each spot on the new arrays in the new slideport, Calibration Wizard (Standard Cellomics Software for instrument calibration) was used. Compartmental Analysis BioApplication (Cellomics) software was used to quantify fluorescent signal. Data was transferred from the Cellomics to EXCEL and eventually to GraphPad for analysis.

After 23 hours, a range of 0 to 175 MCF-7 cells attached to the spots. MCF-7 cells were found to attach to many different matrices at a density between 25-200 cells per matrix spot (300 μm in diameter) when 250K cells were seeded on the array. MCF-7 cells demonstrate differential attachment: Cell seeding density per spot is different depending on the components in the spots printed on the array.

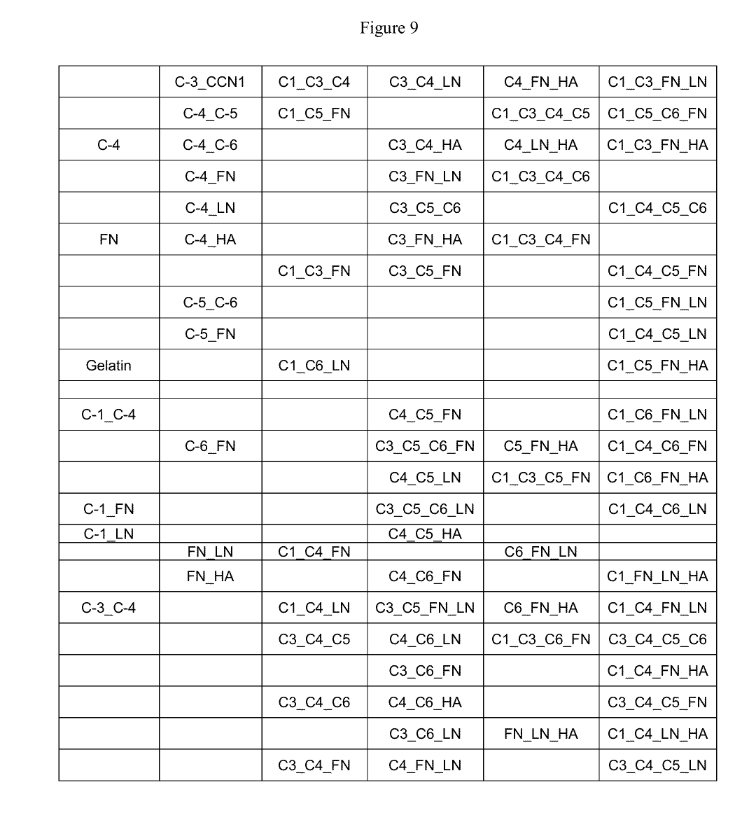

FIG. 9 represents the matrix components and geographical locations of components on the array to which between 25 and 200 cells attached.

For the purpose of this experiment Fibronectin (FN) was used alone as a control matrix to assess paclitaxel cytotoxicity in MCF-7 cells. Fibronectin is a well referenced control matrix for culturing MCF-7 cells in vitro.

MCF-7 cells are sensitive to paclitaxel when attached to fibronectin spots printed on hydrogel. After 16 hrs in the presence of paclitaxel, MCF-7 cells demonstrate a dramatic cytotoxic phenotype when compared to untreated MCF-7 cells.

Using fibronectin as a control matrix for MCF-7 cells, growth data for matrixes that promoted a differential response were then explored. In particular matrices that promoted paclitaxel resistance in MCF-7 cells were sought.

MCF-7 cells grown on a matrix containing collagen IV, collagen V and Fibronectin are observed to maintain a resistance to paclitaxel (as determined by the same cytotoxicity parameters). It appears that the addition of collagen IV and V to fibronectin (or the resulting decrease in fibronectin concentration from 250 μg/ml to 84 μg/ml, because the protein concentration per spot is held constant at 250 μg/ml) promote MCF-7 resistance to paclitaxel.

The preceding discussion and examples demonstrate that invention screening and culturing devices offer a unique platform for screening small numbers of cells (250K in this example) against large numbers of screening components (in this example, 1100) at a microscale level. The platform uses high content imaging and analysis to enable multiparameter readouts on cellular functions and phenotypes. The invention screening platform is amenable to screening a wide variety of drugs that promote certain measurable cellular response.

The examples provided herein illustrate the wide range of uses to which the invention screening platform can be applied, e.g., to identify a number of matrix conditions that promote MCF-7 cell attachment. In addition, invention screening platform can be employed to examine the effects of paclitaxel on MCF-7 cells attached to various matrices. Furthermore, it is useful to identify matrices that contain a combination of ECMPs at equal concentrations that appear to promote a resistance to paclitaxel in MCF-7 cells. This latter observation is an important finding as there is no standard for what media cells should be seeded on during a chemosensitivity experiment looking at efficacy. Indeed, it does not appear to be possible to readily standardize a matrix as it appears that cells grow in many different microenvironments containing many different ECMPs in combination in vivo. Accordingly, the invention screening and culturing platform is a useful tool for providing at least a snapshot of the effects of various ECM and ECMP molecules on cell function in the presence of drugs.

### Example 4

### Use of Invention Device and Methods to Identify Extra Cellular Matrix Protein Combinations that Promote Differentiation of Mesenchymal Stem Cell to Cardiomyocytes

SingleQuots™ (CGM SingleQuot kit, Lonza PT-4105) mesenchymal cell growth supplements, L-glutamine and GA-100 were thawed overnight at 2-8° C. The bottle was aseptically opened and the entire contents were added to the 440 ml mesenchymal stem cell basal medium (MSCBM). This is referred as a human mesenchymal stem cell growth medium (MSCGM), and is stored at 2-8° C. The MSCGM was used within 1 month.

The human Bone Marrow mesenchymal stem cells (Lonza, PT-2501, lot OF4266) were thawed and cultured according to the manufacturer's protocol. The cryovial was quickly thawed in a 37° C. water bath until the last sliver of ice was melted. Using a micropipette, thawed cell suspension was gently transferred into 5 ml MSCGM. Cells then were centrifuged at 500×g for 5 minutes at room temperature.

The pellet was re-suspended in 1 mL of MSCGM by gently pipetting up and down. The cell count was determined with Trypan Blue using a hemacytometer. The recommended seeding density of human mesenchymal stem cells is 5000-6000 viable cells per cm². Cultures were incubated at 37° C., 5% CO₂ and 90% humidity and monitored daily. The medium was replaced every 2-3 days with an equal volume of warm MSCGM™. Cells were 80% confluent by day 5 or 6 and ready to subculture.

To passage cells, media were aseptically removed from the flasks. Adherent cells were washed with PBS twice to remove residual medium. Sufficient volume of 0.25% Trypsin/EDTA (Invitrogen) solution was added to the flask to cover the cell layer (5 ml for T75 flask) and incubated 3-5 min at 37° C. Cultures were observed under the microscope every 2 min to ensure that all cells were detached. Equal volume (5 ml for T75) of warm MSCGM was added to each vessel. Cells were collected into a 50 ml tube; flasks were washed with 5 ml of fresh media to recover remaining cells. To remove the trypsin, cells were centrifuged at approximately 500×g for 5 minutes at RT. Supernatant was removed and the resulting cell pellet was resuspended in 1 ml of MCSGM. Viable cells were counted with Trypan Blue using a hemacytometer. Cells were diluted at a final concentration of 4×10⁴ cells/ml. Cells were used by passage 4.

MicroMatrix™ 96 (MicroStem, Inc.) slides were removed from −20° C. and brought to room temperature (approx. 10 minutes). Using sterile techniques, 4 MicroMatrix™ 96 slides were removed from packaging and consolidated into one 4 chamber plate (Nunc). The slides were washed with sterile PBS by slowly pipetting, being careful not to scrape the surface of the slides.

Five milliliters of mesenchymal stem cell growth media containing 5×10⁵ cells (passage 3) were added to each of 4 MicroMatrix™ 96 slides. The plate containing the slides was placed in a 37° C. incubator with 5% CO₂ and 95% humidity. Cells were allowed to adhere overnight. The following day non adherent/floating cells were removed by media exchange. To induce cardiac differentiation, MSCs were treated with 0, 5 and 10 μM demethylating agent 5′-Azacytidine (Sigma-Aldrich) in media for 24 hrs. Cells on the slides were then fixed and stained with appropriate antibodies for early cardiomyocytes expression markers.

Human Bone marrow mesenchymal stem cells were stained with CD29 and Nkx2.5. CD29 is one of the essential surface molecules expressed on human BM MSC. Nkx2.5 is an early cardiomyocyte-specific transcription factor involved in cardiomyocyte differentiation.

The media from 5′-Azacytidine treated and non-treated (control) samples were aspirated and slides were gently washed with PBS. Cells were then fixed with 5 ml of 4% PFA for 15 min at room temperature (RT). Cells on the slides were washed again three times with 4 ml PBS and then permeabilized with 0.1% Triton X-100 10 min at RT. Non specific protein-protein interactions were blocked with 2% BSA for 20 min. Mouse anti-Human CD29 and rabbit anti-human Nkx2.5 antibodies at 1:100 dilutions each were added to each slide and incubated for at least 1 hr at RT. Slides were washed three times with PBS. A goat anti-mouse Alexa Flour 488 secondary antibody (1:400 dilution) and donkey anti rabbit Alexa Flour 647 (1:400) were used for immunofluorescent detection (Invitrogen). Slides were washed three times with PBS and cell nuclei were labeled with Hoechst 33342 (Invitrogen).

Slides were placed in the MicroStem SlideHolder™ for imaging on the Cellomics vTI Arrayscan™ (Thermo Fisher). Brightfield images were obtained using a Leica microscope with a camera attached.

The MicroStem SlideHolder™ containing 4 processed MicroMatrix™ 96 slides were placed in the Cellomics. The instrument was set up to capture images at 3 different wave lengths (357 nm, 488 nm, and 647 nm). Each of the 9 replicated conditions was captured at 5× and 20× magnifications. Using the cell compartmentalization Bio-application software, cells attached to spots were segmented and identified by nucleus. Cell adherence was determined by the number of cells present on a spot.

In accordance with the present invention, it has been found that selective extracellular matrix proteins alone or in various combinations support differentiation of MSCs towards cardiogenic lineage, indicated by increase in Nkx.2.5 expression. In contrast, cells exposed to other microenvironments exhibited no detectable changes in Nkx2.5 expression. Meanwhile, CD 29 expression maintained unchanged overall after 5′-azacytidine treatment regardless of ECMP conditions.

The examples provided herein illustrate the wide range of uses to which the invention screening and culturing devices can be applied, e.g., to identify a number of matrix conditions that promote MSCs attachment. In addition, invention screening and culturing devices can be employed to examine the effects of 5′-azacytidine on MSCs attached to various matrices. Furthermore, it is useful to identify matrices that contain a combination of ECMPs that appear to promote directed differentiations of MSCs to cardiomyocyates in the presence of 5′-azacytidine. This latter observation is an important finding as there is no standard for what media cells should be seeded on during a differentiation process. Indeed, it is impossible to standardize a matrix as it appears that cells grow in many different microenvironments containing many different ECMPs in combination in vivo. Accordingly, the invention screening platform is a useful tool for providing at least a snapshot of the effects of various ECM and ECMP molecules on stem cell differentiation process(es).

### Example 5

### Use of Invention Device and Methods to Identify Extra Cellular Matrix Protein Combinations that Influence epithelial Mesenchymal Transition of Cancer cells during Tumor Invasion and Metastasis

Packages containing products which embody present invention (MicroMatrix™ slides) were removed from −20° C. and brought to room temperature (RT). Using sterile techniques, 4 MicroMatrix™ slides were removed from packaging and put into a 4-chamber slide tray. A549 cells (ATCC, Rockville, Md.) were cultured according to manufacturer's protocol in complete medium of DMEM containing 10% FBS and antibiotics (Life Technology, CA) in standard tissue culture treated T-75 flasks (Corning).

Cells were trypsinized with EDTA for 5 minutes and neutralized with 10 ml of complete medium. Neutralized cell suspensions were put into a 50 ml conical tube and centrifuged for 6 minutes at 1100 rpm. Five milliliters of complete medium containing 5×10⁵ cells was added to each of 4 identical MicroMatrix™ slides in the slide tray. The tray was placed in a 37° C. incubator with 5% CO₂ and cells were allowed to attach to ECM spots overnight (14 hrs).

Unattached cells and media were aspirated from the slides and 5 ml of media containing 1% FBS with or without 5 ng/ml of TGF-β were added to each chamber. After 24 hrs, media was aspirated from the chamber and 5 ml of 4% para-formaldehyde (PFA) in 1×PBS (fixation buffer) was added to each slide for 10 minutes at RT.

Fixation buffer was aspirated from chambers and fixed slides were stained for nuclei, e-cadherin and vimentin expression. Briefly, mouse anti-human e-cadherin antibody (BD, CA) and rabbit anti-human vimentin (Cell Signaling Technology) antibody were added to each of 4 slides at a 1:100 dilution in PBS at RT for 3 hours. Primary antibodies were aspirated from slides, and slides were washed 2 times with PBS. After washing, 5 ml of secondary antibody solution containing chicken anti-mouse Alexa 488 (Invitrogen, CA) (1:400) and goat anti-rabbit Alexa 647 (Invitrogen, CA) (1:400) as well as Hoescht dye (Invitrogen, CA) (1 ug/ml) in 1×PBS were added to each slide.

After staining, slides were washed 4 times with PBS and allowed to dry in the 4-chamber slide tray. Slides were placed in a MicroStem SlideHolder™ for imaging on a Cellomics vTI Arrayscan™ (Thermo Fisher, Pittsburgh, Pa.). Brightfield images were obtained using a standard Nikon light microscope with a camera attached.

The MicroStem SlideHolder™ containing 4 processed MicroMatrix™ slides were placed in the Cellomics. The MicroMatrix™ standard form factor was used to capture images automatically at 3 different wavelengths using the Hoescht, FITC and CY5 channels. In this case, form factors were developed by defining each block of 3×3 spots (9 replicates for each condition) as a “well” and each spot was defined as a “field”. Each of 9 replicated conditions was captured at 5× and 20× magnification. Using the Cell Compartmentalization Bio-application software, cells attached to spots were segmented and identified by nuclei in the Hoescht channel.

Cell adhesion was determined by the number of nuclei present on a spot. Cell adherence was ECM composition dependent regardless of the presence or absence of TGF-β. Therefore, not all spots demonstrated cell adherence. Using the Cellomics software, a threshold of 25 cells per spot was created as the minimum adherence required to be considered for further analysis. Cells adhered to ECM compositions that met the 25 cell/spot criteria were then analyzed for e-cadherin and vimentin expression using the same Bioapplication software. A ring was formed around segmented objects (nuclei) and fluorescence measurements for secondary channels were captured. Average intensities of e-cadherin and vimentin were calculated and normalized by cells per spot.

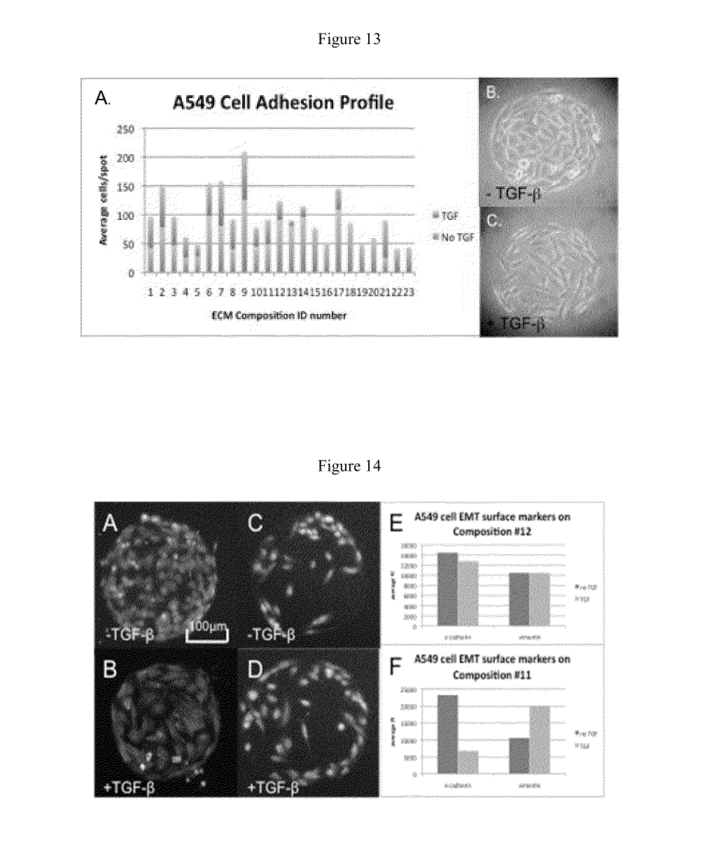

A549 cells demonstrated preferential attachment and distinct adherence morphologies to certain ECM combinations. In conducting the experiments described herein, it was observed that certain ECM compositions promoted cell attachment of A549 cells in the absence of TGF-β. However, cell detachment is observed upon addition of TGF-β, most likely due to the biological changes that occur in cells during EMT (FIG. 13).

Combinations of ECMs showed enhancement of TGF-β induced EMT. A549 cells attached to certain combinations of ECMs for 24 hrs in the presence of TGF-β demonstrated EMT related decreases in e-cadherin and increases in vimentin expression when compared to those from other ECM compositions. Conversely, some ECM compositions appeared to limit A549 cell transformation in the presence of TGF-β, further demonstrating the importance of cell-matrix interactions and its ability to dictate cellular fate and function in an in vitro setting. In this instance, MicroMatrix™ was used to identify a set of conditions that not only provides physiologically relevance but also enhances the experimental window for testing. ECM composition #11 identified using the MicroMatrix™ product demonstrates the desired phenotypic effect and may be used as a model for further analysis (FIG. 14).

Although the invention has been described with reference to embodiments and examples, it should be understood that various modifications can be made without departing from the spirit of the invention.

All references cited herein are hereby expressly incorporated by reference in their entireties. Where reference is made to a uniform resource locator (URL) or other such identifier or address, it is understood that such identifiers can change and particular information on the internet can be added, removed, or supplemented, but equivalent information can be found by searching the internet. Reference thereto evidences the availability and public dissemination of such information.

1. A combinatorial extracellular matrix (ECM) screening and culturing device comprising a support coated with a hydrogel, on which a plurality of spots comprising one or more ECM components are printed thereon,
wherein:
the concentration of ECM component(s) per spot falls in the range of about 0.01 mg/ml up to about 1 mg/ml,
each ECM component is printed in replicates of at least 3 up to about 20,
the resulting ECM spots have a minimum diameter to allow attachment of at least one cell thereto (typically in the range of about 50 up to 1000 μm), and
the center-to-center distance between spots is sufficient to preclude overlap of the spots (typically the distance is at least 100 μm).

2. The device of claim 1 wherein said complex microenvironment comprises two or more components selected from the group consisting of extracellular matrix proteins or components thereof, cellular adhesion molecules, monosaccharides, oligosaccharides, polysaccharides, glycoproteins, proteoglycans, non-proteoglycan polysaccharides, cell communication molecules, complex carbohydrates, lipids, vitamins and metabolites thereof, naturally occurring low molecular weight biologically active molecules, synthetic low molecular weight biologically active molecules, polypeptides, synthetic polymers, biopolymers, antibodies, nucleic acids, inorganic salts, and media supplements.

3. A method of making a screening and culturing device according to claim 1, said method comprising printing a plurality of ECM components on a suitable support material.

4. A method of screening a plurality of extracellular matrix (ECM) components to identify those which support cell viability, growth and/or proliferation, and transformation and/or differentiation, said method comprising:
applying cells to a screening and culturing device according to claim 1,
assaying cell morphology and/or behavior upon incubation of said cell, and
identifying those ECM components which support cell viability, growth and/or proliferation, and transformation and/or differentiation.

5. A method of culturing cells on a plurality of extracellular matrix (ECM) components which support cell viability, growth and/or proliferation, said method comprising:
applying cells to a screening and culturing device according to claim 1,
assaying cell morphology and/or behavior upon incubation of said cell, and
identifying those ECM components which support cell viability, growth and/or proliferation.
